(19) United States
(12) Patent Application Publication
(10) Pub. No.: US 2004/0242498 A1
(43) Pub. Date: Dec. 2, 2004

(54) CXCR3 antagonists

(76) Inventors: Tassie L. Collins, San Mateo, CA (US); Michael G. Johnson, San Francisco, CA (US); Ji Ma, Foster City, CA (US); Julio C. Medina, San Carlos, CA (US); Shichang Miao, Foster City, CA (US); George R. Tonn, San Carlos, CA (US); manfred schneider, brooklyn, NY (US)

Correspondence Address: JONES DAY, 222 EAST 41ST ST, NEW YORK, NY, 10017, US

(21) Appl. No.: 10/789,165

(22) Filed: Feb. 26, 2004

Related U.S. Application Data
(60) Provisional application No. 60/451,157, filed on Feb. 27, 2003.

(51) Int. Cl. A61K 31/7052; C07D 487/02; A61K 31/519

(52) U.S. Cl. 514/23; 514/264.1; 544/280; 536/17.4

(57) ABSTRACT

Compounds, compositions and methods that are useful in the treatment of inflammatory and immune conditions and diseases are provided herein. In particular, the invention provides compounds which modulate the expression and/or function of a chemokine receptor. The subject methods are useful for the treatment of inflammatory and immunoregulatory disorders and diseases, such as multiple sclerosis, rheumatoid arthritis and type I diabetes.

### 1. FIELD OF THE INVENTION

The present invention relates to novel antagonists of the CXCR3 receptor, compositions comprising the novel compounds and methods of their use for the treatment of, for example, inflammatory and immunoregulatory disorders and diseases, including asthma and allergic diseases, as well as autoimmune pathologies such as rheumatoid arthritis and atherosclerosis. Certain of the novel antagonists are metabolites of previously reported antagonists of the CXCR3 receptor.

### 2. BACKGROUND OF THE INVENTION

Chemokines are chemotactic cytokines that are released by a wide variety of cells to attract macrophages, T cells, eosinophils, basophils and neutrophils to sites of inflammation (reviewed in Schall, Cytokine, 3:165-183 (1991), Schall, et al., Curr. Opin. Immunol., 6:865-873 (1994) and Murphy, Rev. Immun., 12:593-633 (1994)). In addition to stimulating chemotaxis, other changes can be selectively induced by chemokines in responsive cells, including changes in cell shape, transient rises in the concentration of intracellular free calcium ions ([Ca²⁺])ᵢ, granule exocytosis, integrin upregulation, formation of bioactive lipids (e.g., leukotrienes) and respiratory burst, associated with leukocyte activation. Thus, the chemokines are early triggers of the inflammatory response, causing inflammatory mediator release, chemotaxis and extravasation to sites of infection or inflammation.

There are four classes of chemokines, CXC(α), CC(β), C(γ), and CX₃C (δ), depending on whether the first two cysteines are separated by a single amino acid (C-X-C), are adjacent (C-C), have a missing cysteine pair (C), or are separated by three amino acids (CX₃C). The α-chemokines, such as interleukin-8 (IL-8), melanoma growth stimulatory activity protein (MGSA), and stromal cell derived factor 1 (SDF-1) are chemotactic primarily for neutrophils and lymphocytes, whereas β-chemokines, such as RANTES, MIP-1α, MIP-1β, monocyte chemotactic protein-1 (MCP-1), MCP-2, MCP-3 and eotaxin are chemotactic for macrophages, T-cells, eosinophils and basophils (Deng, et al., Nature, 381:661-666 (1996)). The C chemokine lymphotactin shows specificity for lymphocytes (Kelner, et al., Science, 266:1395-1399 (1994)) while the CX₃C chemokine fractalkine shows specificity for lymphocytes and monocytes (Bazan, et al., Nature, 385:640-644 (1997)).

Chemokines bind specific cell-surface receptors belonging to the family of G-protein-coupled seven-transmembrane-domain proteins (reviewed in Horuk, Trends Pharm. Sci., 15:159-165 (1994)) termed “chemokine receptors.” On binding their cognate ligands, chemokine receptors transduce an intracellular signal through the associated heterotrimeric G protein, resulting in a rapid increase in intracellular calcium concentration. There are at least twelve human chemokine receptors that bind or respond to β-chemokines with the following characteristic pattern: CCR1 (or “CKR-1” or “CC-CKR-1”) MIP-1α, MIP-1β, MCP-3, RANTES (Ben-Barruch, et al., J. Biol. Chem., 270:22123-22128 (1995); Neote, et al., Cell, 72:415-425 (1993)); CCR2A and CCR2B (or “CKR-2A”/“CKR-2A” or “CC-CKR-2A”/“CC-CKR2A”) MCP-1, MCP-3, MCP-4; CCR3 (or “CKR-3” or “CC-CKR-3”) eotaxin, RANTES, MCP; (Ponath, et al., J. Exp. Med., 183:2437-2448 (1996)); CCR4 (or “CKR-4” or “CC-CKR-4”) TARC, MDC (Imai, et al., J. Biol. Chem., 273:1764-1768 (1998)); CCR5 (or “CKR-5” or “CC-CKR-5”) MIP-1α, RANTES, MIP-1β (Sanson, et al., Biochemistry, 35:3362-3367 (1996)); CCR6 MIP-3 alpha (Greaves, et al., J. Exp. Med., 186:837-844 (1997)); CCR7 MIP-3 beta and 6Ckine (Campbell, et al., J. Cell. Biol., 141:1053-1059(1998)); CCR8 I-309, HHV8 vMIP-I, HHV-8 vMIP-II, MCV vMCC-I (Dairaghi, et al., J. Biol. Chem., 274:21569-21574 (1999)); CCR9 TECK (Zaballos, et al., J. Immunol., 162:5671-5675 (1999)), D6 MIP-1 beta, RANTES, and MCP-3 (Nibbs, et al., J. Biol. Chem., 272:32078-32083 (1997)), and the Duffy blood-group antigen RANTES, MCP-1 (Chaudhun, et al., J. Biol. Chem., 269:7835-7838 (1994)).

Chemokine receptors, such as CCR1, CCR2, CCR2A, CCR2B, CCR3, CCR4, CCR5, CCR6, CCR7, CCR8, CCR9, CXCR1, CXCR2, CXCR3, CXCR4, CXCR5, CX₃CR1, and XCR1 have been implicated as being important mediators of inflammatory and immunoregulatory disorders and diseases, including asthma and allergic diseases, as well as autoimmune pathologies such as rheumatoid arthritis and atherosclerosis.

The CXCR3 chemokine receptor is expressed primarily in T lymphocytes, and its functional activity can be measured by cytosolic calcium elevation or chemotaxis. The receptor was previously referred to as GPR9 or CKR-L2. Its chromosomal location is unusual among the chemokine receptors in being localized to Xq13. Ligands that have been identified that are selective and of high affinity are the CXC chemokines, IP10, MIG and ITAC.

The highly selective expression of CXCR3 makes it an ideal target for intervention to interrupt inappropriate T cell trafficking. The clinical indications for such intervention are in T-cell mediated autoimmune diseases such as multiple sclerosis, rheumatoid arthritis, and type I diabetes. Inappropriate T-cell infiltration also occurs in psoriasis and other pathogenic skin inflammation conditions, although the diseases may not be true autoimmune disorders. In this regard, up-regulation of IP-10 expression in keratinocytes is a common feature in cutaneous immunopathologies. Inhibition of CXCR3 can be beneficial in reducing rejection in organ transplantation. Ectopic expression of CXCR3 in certain tumors, especially subsets of B cell malignancies, indicates that selective inhibitors of CXCR3 will have value in tumor immunotherapy, particularly attenuation of metastasis.

In view of the clinical importance of CXCR3, compounds that modulate CXCR3 function can be used for the development of new therapeutic agents. Compounds that are potent antagonists of CXCR3 have been described in U.S. Patent Publication 2002/0169159A1. One of these promising antagonists is in clinical development for therapeutic treatment of inflammation in conditions such as rheumatoid arthritis, inflammatory bowel disease and psoriasis. Development of the antagonist could yield an oral therapy to treat these chronic illnesses.

Methods of administering such antagonists to maximize their bioavailabilty and therapeutic activity are needed to develop treatments of, for example, inflammatory and immunoregulatory disorders and diseases, including asthma and allergic diseases, as well as autoimmune pathologies such as rheumatoid arthritis and atherosclerosis.

### 3. SUMMARY OF THE INVENTION

The present invention is based, in part, on the discovery that when a potent CXCR3 antagonist is administered to a subject, it is converted into one or more metabolites that are effective to modulate CXCR3 function. The present invention provides compounds which are useful in the treatment or prevention of certain inflammatory and immunoregulatory disorders and diseases, including asthma and allergic diseases, as well as autoimmune pathologies such as rheumatoid arthritis and atherosclerosis.

In one aspect, the present invention provides compounds having the following general formula (I):

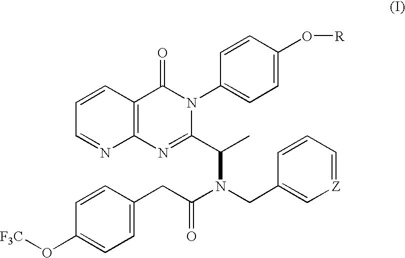

wherein Z is N or N⁺—O⁻; and R is hydrogen, (C₁-C₂₀)alkyl, (C₂-C₂₀)heteroalkyl, heteroaryl, aryl, heteroaryl(C₁-C₆)alkyl, heteroaryl(C₂-C₆)heteroalkyl, aryl(C₁-C₆)alkyl or aryl(C₂-C₆)heteroalkyl; with the proviso that when Z is N, R is not (C₁-C₂)alkyl, (C₂)heteroalkyl or aryl(C₁-C₃)alkyl. In certain embodiments, R is hydrogen or a glycosyl such as glucuronyl. Unless otherwise indicated, the compounds provided in the above formula are meant to include pharmaceutically acceptable salts or prodrugs thereof.

In another aspect, the present invention provides pharmaceutical compositions comprising a compound of formula (I) and a pharmaceutically acceptable excipient or carrier.

In a further aspect, the present invention provides methods for the treatment or prevention of an inflammatory or immune condition or disorder, comprising administering to a subject in need of such treatment or prevention a therapeutically effective amount of a compound of formula (I). Preferred subjects for the methods of the invention include mammals such as humans.

In certain embodiments, the present invention provides methods for the treatment or prevention of, for example, an inflammatory or immune condition or disorder, comprising administering to a subject in need thereof an amount of a parent compound that provides the subject with an effective amount of a compound of formula (I). The preferred parent compound is compound 101, described in detail below.

The present invention also provides methods for the treatment or prevention of a condition or disorder mediated by the CXCR3 chemokine receptor, comprising administering to a subject in need of such treatment or prevention a therapeutically effective amount of a compound of formula (I).

The present invention also provides methods for the modulation of CXCR3, comprising contacting a cell with a compound of formula (I).

The present invention further provides methods for the modulation of CXCR3, comprising contacting a CXCR3 protein with a compound of formula (I).

In addition, the present invention provides methods of making compounds of formula (I).

### 4. BRIEF DESCRIPTION OF THE DRAWINGS

FIG. 1 provides the structures of compounds 101, 103, 105, 107, 109, 111;

FIG. 2 provides an exemplary scheme for the synthesis of compound 101; and

FIG. 3 illustrates the metabolism of compound 101 in several mammalian subjects.

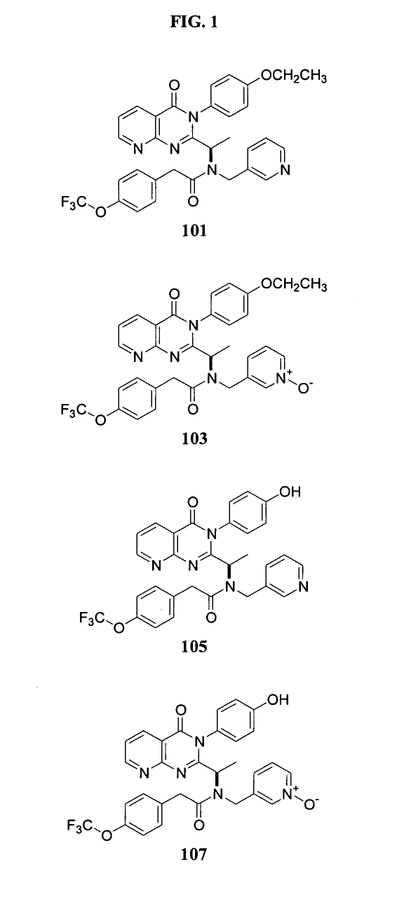

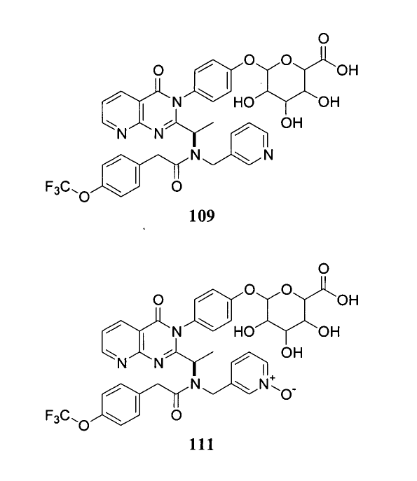

### 5. DETAILED DESCRIPTION OF THE INVENTION

### 5.1 Definitions

As used herein, the term “active” means effective to modulate, e.g., inhibit, CXCR3 function.

The term “parent compound”, as used herein, refers to any compound that can be converted to one or more compounds of the invention when administered to a subject. A parent compound may be active (e.g., a drug) or inactive (e.g., a prodrug). In preferred embodiments, the parent compound is compound 101. In particularly preferred embodiments, the compounds of the invention are active.

The term “metabolite”, as used herein, refers to a chemically modified form of compound 101 that can be found in vivo when compound 101 is administered to a subject. Modifications include, but are not limited to, oxidation, reduction and hydrolysis of a functional group and conjugation to, e.g., glucuronic acid, glutathione, a sulfate, an amino acid, a peptide or an acetyl group. The subject can be an animal or mammal such as a rat, dog, monkey or, preferably, a human. The therapeutic effect(s) that is observed when a parent compound, e.g., compound 101, is administered to a subject may result from chemokine receptor modulation by the parent compound and/or one or more metabolites thereof. Thus, in certain embodiments, the present invention provides methods of administering a parent compound to a subject to provide the subject with an effective amount of a metabolite.

The terms “treat”, “treating” or “treatment”, as used herein, refer to a method of alleviating or abrogating a disease and/or its attendant symptoms. The terms “prevent”, “preventing” or “prevention”, as used herein, refer to a method of barring a subject from acquiring a disease.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkylene” by itself or as part of another substituent means a divalent radical derived from an alkane, as exemplified by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively. Similarly, the term dialkylamino refers to an amino group having two attached alkyl groups that can be the same or different.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃—)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. When a prefix such as (C₂-C₈) is used to refer to a heteroalkyl group, the number of carbons (2-8, in this example) is meant to include the heteroatoms as well. For example, a C₂-heteroalkyl group is meant to include, for example, —CH₂OH (one carbon atom and one heteroatom replacing a carbon atom) and —CH₂SH. The term “heteroalkylene” by itself or as part of another substituent means a divalent radical derived from heteroalkyl, as exemplified by —CH₂—CH₂—S—CH₂CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include trifluoromethyl, 2,2,2-trifluoroethyl, 4-chlorobutyl, 3-bromopropyl, and the like.

The term “aryl” means, unless otherwise stated, a polyunsaturated, typically aromatic, hydrocarbon substituent which can be a single ring or multiple rings (up to three rings) which are fused together or linked covalently. The term “heteroaryl” refers to aryl groups (or rings) that contain from zero to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —CN and —NO₂ in a number ranging from zero to (2m+1), where m is the total number of carbon atoms in such radical. R′, R″ and R′″ each independently refer to H, unsubstituted (C₁-C₈)alkyl and heteroalkyl, unsubstituted aryl, aryl substituted with 1-3 halogens, alkoxy or thioalkoxy groups, or aryl-(C₁-C₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. From the above discussion of substituents, one of skill in the art will understand that the term “alkyl” in its broadest sense is meant to include groups such as haloalkyl (e.g., —CF₃ and —CH₂CF₃) and acyl (e.g., —C(O)CH₃, —C(O)CF₃, —C(O)CH₂OCH₃, and the like). Preferably, the alkyl groups will have from 0-3 substituents, more preferably 0, 1, or 2 substituents, unless otherwise specified.

Similarly, substituents for the aryl and heteroaryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from H, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣB—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted (C₁-C₆)alkyl.

As used herein, the term “heteroatom” is meant to include oxygen (O), nitrogen (N), sulfur (S) and silicon (Si).

The term “glycosyl” refers to a saccharide or saccharose radical. Examples of glycosyl groups of the invention include galactosyl, glucuronyl, deoxy-glucosyl, iduronyl, glucosyl, N-acetyl glucosaminosyl, fructosyl, sialosyl, hyaluronosyl, sedoheptulosyl, xylulosyl, ribulosyl, ribosyl, xylitosyl, daunosaminosyl, arabinosyl, fucosyl, deoxy-ribosyl, mannosyl, N-acetyl-galactosyl, rhamnosyl, 3,6-anhydrogalactosyl, sialylfucosyl, and xylosyl.

The term “pharmaceutically acceptable salts” is meant to include salts of the compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, et al. (1977) J. Pharm. Sci. 66:1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the active compounds described herein are inactive compounds that readily undergo chemical changes under physiological conditions to provide active compounds of the present invention. Additionally, prodrugs can be converted to active compounds of the present invention by chemical or biochemical methods in an ex vivo environment. For example, prodrugs can be slowly converted to active compounds of the present invention when placed in a transdermal patch reservoir with a suitable enzyme or chemical reagent. Prodrugs are often useful because, in some situations, they may be easier to administer than the active compound. They may, for instance, be bioavailable by oral administration whereas the active compound is not. The prodrug may also have improved solubility in pharmacological compositions over the active compound. A wide variety of prodrug derivatives are known in the art, such as those that rely on hydrolytic cleavage or oxidative activation of the prodrug. An example, without limitation, of a prodrug would be a compound of the present invention which is administered as an ester (the “prodrug”), but then is metabolically hydrolyzed to the carboxylic acid, the active entity. Additional examples include peptidyl derivatives of an active compound of the invention.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, enantiomers, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). Radiolabeled compounds are useful as therapeutic agents, e.g., cancer therapeutic agents, research reagents, e.g., binding assay reagents, and diagnostic agents, e.g., in vivo imaging agents. All isotopic variations of the compounds of the present invention, whether radioactive or not, are intended to be encompassed within the scope of the present invention.

### 5.2 Embodiments of the Invention

The present invention is directed to compounds, compositions and methods useful in the modulation of chemokine receptor activity, particularly CXCR3. The compounds of the invention are based, in part, on the discovery that a potent antagonist of CXCR3 is converted by subjects such as rats, dogs, monkeys and humans into one or more metabolites that are effective to modulate, e.g., antagonize, CXCR3. Like the potent CXCR3 antagonist, the compounds of the invention are useful for the treatment of, for example, inflammatory and immunoregulatory disorders, and can be administered directly to subjects, e.g., humans, as formulated pharmaceuticals. The compounds of the invention are also useful for identifying and/or designing compounds that modulate CXCR3 function, e.g., CXCR3 antagonists, and compounds that are converted to one or more compounds that modulate CXCR3 function under physiological conditions. The compounds of the invention can be administered to a subject directly or via the administration of a parent compound. For instance, a parent compound of a compound of the invention can be administered to a subject to provide the subject with an effective amount of the compound of the invention according to the methods described in detail below. Specific doses or dosing regimens of the parent compound to achieve certain levels of the metabolite are also encompassed herein. These doses or dosing regimens and methods encompass the treatment or prevention or disease while reducing or avoiding unwanted or adverse side effects. In certain embodiments, the administration of the isolated or purified metabolite to a subject in need thereof is also provided.

The compounds of the present invention are those which inhibit at least one function or characteristic of a mammalian CXCR3 protein, for example, a human CXCR3 protein. The ability of a compound to inhibit such a function can be demonstrated in a binding assay (e.g., ligand binding or agonist binding), a signaling assay (e.g., activation of a mammalian G protein, induction of rapid and transient increase in the concentration of cytosolic free calcium), and/or cellular response function (e.g., stimulation of chemotaxis, exocytosis or inflammatory mediator release by leukocytes). Exemplary assays are described in the Examples below and in U.S. patent application No. U.S. 2002/0169159 A1, the contents of which are hereby incorporated by reference in their entirety.

Compounds

The present invention provides compounds that are useful as antagonists of CXCR3, having particular utility for the treatment or prevention of inflammatory or immune conditions or disorders. The compounds are based, in part, on metabolites of a potent CXCR3 antagonist having the following structure:

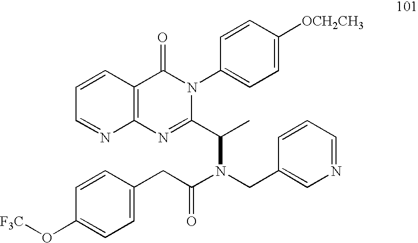

Compound 101 is a potent antagonist of the CXCR3 receptor described in U.S. patent application Publication No. U.S. 2002/0169159 A1, the contents of which are hereby incorporated by reference in their entirety. The therapeutic effect(s) that is observed when compound 101 is administered to a subject may result from chemokine receptor modulation by compound 101 and/or one or more metabolites thereof.

In one aspect, the present invention provides compounds that have the general formula (I):

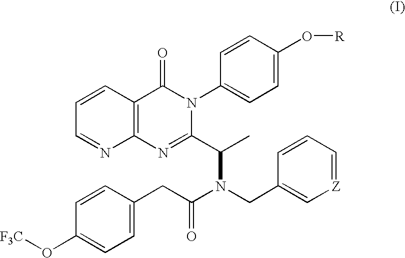

wherein Z is N or N⁺—O⁻; and R is (C₁-C₂₀)alkyl, (C₂-C₂₀)heteroalkyl, heteroaryl, aryl, heteroaryl(C₁-C₆)alkyl, heteroaryl(C₂-C₆)heteroalkyl, aryl(C₁-C₆)alkyl or aryl(C₂-C₆)heteroalkyl; with the proviso that when Z is N, R is not —CH₂—CH₃.

In certain embodiments, Z is N, and R is hydrogen or a glycosyl such as glucuronyl. In other embodiments, Z is N⁺—O⁻, and R is hydrogen or a glycosyl such as glucuronyl.

In embodiments where R is a glycosyl, R can be any glycosyl radical known to those of skill in the art. Preferred are glycosyl radicals known to those of skill in the art to be found in metabolized compounds in subjects in need of treatment for, for example, inflammatory or immune disorders or conditions. In a particular embodiment, the glycosyl is glucuronyl.

The compounds of the invention may display one or more chemical, clinical and/or pharmacological properties, e.g., chemical stability, drug interactions, therapeutic index, side effects profile, absorption, oral bioavailability, distribution, clearance, plasma concentration, serum concentration and toxicity, that may be different from those of compound 101. Such properties may be desirable in, e.g., the treatment of certain disease states and/or patient populations and certain formulations and/or drug combinations.

Exemplary compounds of the invention include:

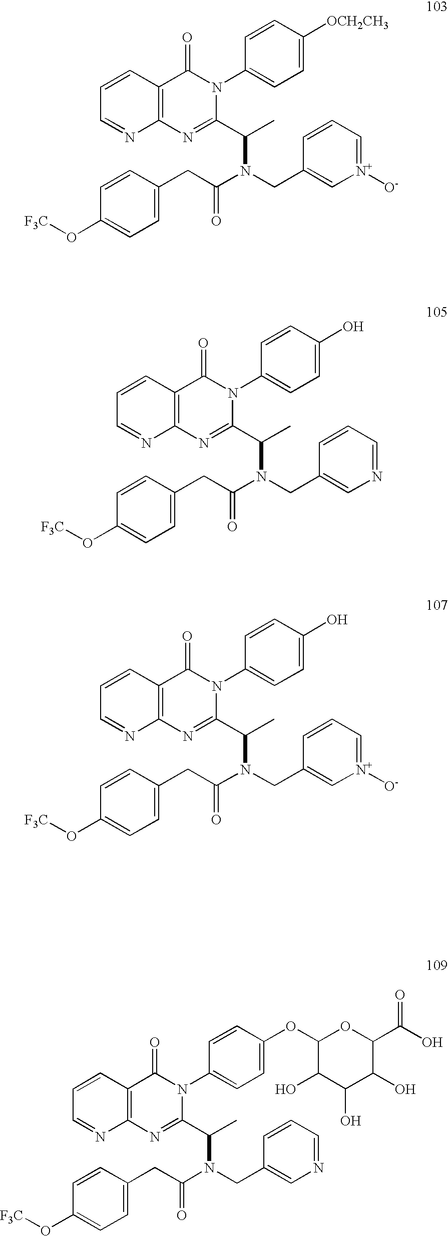

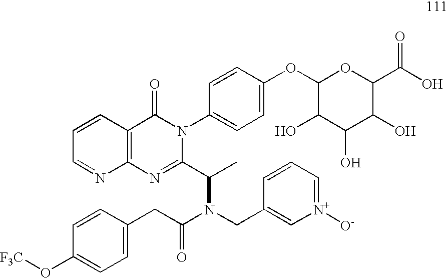

### 5.3 Preparation of the Compounds

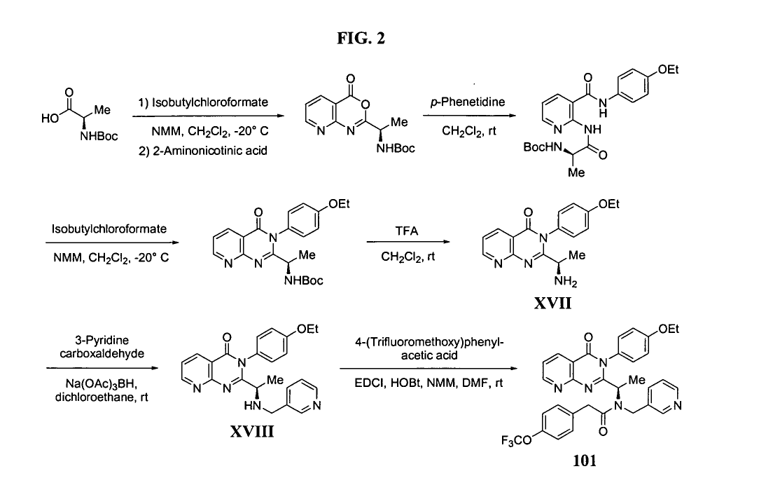

The compounds of the invention can be prepared by a variety of synthetic or semisynthetic techniques. Many of the compounds can be made using compound 101 as a starting material. Compound 101 can be prepared by techniques known or apparent to those of skill in the art. For instance, compound 101 can be prepared according to the methods of U.S. patent application Publication No. U.S. 2002/0169159 A1, the contents of which are hereby incorporated by reference in their entirety. FIG. 2 provides a synthetic scheme for the preparation of compound 101, and the synthesis of compound 101 is described in detail in the examples below. One of skill in the art will appreciate that the substituents can be added or altered before, during or after preparation of the heterocyclic scaffolding and that suitable adjustments in conditions (e.g., temperatures, solvents, etc.) can be made. Additionally, one of skill in the art will recognize that protecting groups may be necessary for the preparation of certain compounds and will be aware of those conditions compatible with a selected protecting group.

One of skill in the art will recognize that any of the compounds of the invention can be obtained by contacting compound 101 with a viable tissue fraction of a subject, for example liver microsomes, and isolating the compound. For instance, compound 103 has been observed when contacting compound 101 with human, monkey, dog and rat liver microsomes. Compound 105 has been observed when contacting compound 101 with rat, human, monkey and dog liver microsomes. Compound 107 has been observed when contacting compound 101 with human and monkey liver microsomes. Compound 109 has been observed when contacting compound 101 with monkey and dog liver hepatocytes.

The compounds of the invention can also be conveniently prepared by synthetic or semisynthetic means. For example, compound 103 can be prepared from compound 101 by oxidizing the pyridyl nitrogen of compound 101 with a suitable oxidizing reagent known to those of skill in the art. Reagents useful for the oxidation of a pyridyl nitrogen include H₂O₂, perbenzoic acid, monoperphthalic acid, peracetic acid, dimethyldioxirane, bis(trimethylsilyl)peroxide, peroxomonosulfuric acid, m-chloroperoxybenzoic acid, sodium perborate monohydrate, potassium peroxymonosulfate and magnesium monoperoxy-phthalate.

Compound 105 can be prepared by following the synthesis of compound 101 in FIG. 2 and Example 1 substituting 4-hydroxyaniline, or a protected version thereof, for p-phenetidine. Alternatively, compound 105 can be synthesized from compound 101 by any suitable dealkylation reaction known to those of skill in the art. Examples include reaction with acidic reagents including Bronsted acids such as hydrobromic acid, hydriodic acid, and trifluoroacetic acid, Lewis acids such as boron tribromide and aluminum chloride (singly or in combination with alkyl sulfurs), pyridine hydrochloride, and hydrobromic acid-acetic acid solution; alkaline reagents such as sodium methoxide, sodium cyanide, lithium diphenylphosphine, and lithium chloride; silicon reagents such as trimethylsilyl iodide; and hydrogenation reduction such as catalytic reduction.

Compound 107 can be prepared by dealkylation of compound 103 according to any of the dealkylation methods discussed above. Alternatively, compound 107 can be prepared by oxidation of compound 105 according to any of the methods for oxidizing a pyridyl nitrogen discussed above.

Compounds 109 and 111 can be prepared by glucuronidation of compounds 105 and 107, respectively. Any suitable method of glucuronidation known to those of skill in the art can be used. Examples can be found in Kaspersen et al., Xenobiotica 17:1451-1471 (1987).

Compounds according to formula (I) can be prepared according to similar techniques. For instance, a compound according to formula (I) can be prepared by following the synthesis of compound 101 as described in FIG. 2 and in the examples below. One of skill in the art need only substitute a suitable reactant for p-phenetidine in the synthesis to yield the R group of the final compound or a precursor thereof. Such substitutions will be readily apparent to those of skill in the art. In certain embodiments where R is a glycosyl group, the compound can be prepared by synthesis of compound 105 as described above followed by a glycosylation reaction known to those of skill in the art.

In embodiments where Z is N⁺—O⁻, the synthesis of a compound according to formula (I) can include a suitable oxidation step such as those discussed above to yield the appropriate pyridyl-N-oxide group.

The exemplary methods and the examples described herein are illustrative of the present invention and are not to be construed as limiting the scope thereof. For example, the methods used to prepare the exemplary compounds of the invention may produce additional compounds.

### 5.4 Compositions

In another aspect, the present invention provides pharmaceutical compositions for modulating chemokine receptor activity in humans and animals. The compositions comprise a compound of the present invention with a pharmaceutically acceptable carrier or diluent. In certain methods of the invention described below, the pharmaceutical compositions comprise compound 101.

“Modulation” or modulating of chemokine receptor activity, as used herein in its various forms, is intended to encompass antagonism, agonism, partial antagonism and/or partial agonism of the activity associated with a particular chemokine receptor, preferably the CXCR3 receptor. The term “composition” as used herein is intended to encompass a product comprising the specified ingredients (and in the specified amounts, if indicated), as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The pharmaceutical compositions for the administration of the compounds of this invention may conveniently be presented in unit dosage form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108; 4,166,452 and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. As used herein, topical application is also meant to include the use of mouth washes and gargles.

The pharmaceutical composition and method of the present invention may further comprise other therapeutically effective compounds as noted herein which are usually applied in the treatment or prevention of the above mentioned pathological conditions.

### 5.5 Methods of Use

In yet another aspect, the present invention provides methods of treating CXCR3-mediated conditions or diseases by administering to a subject having such a disease or condition, a therapeutically effective amount of compound or composition of the invention. The “subject” is defined herein to include animals such as mammals, including, but not limited to, primates (e.g., humans), cows, sheep, goats, horses, dogs, cats, rabbits, rats, mice and the like.

As used herein, the phrase “CXCR3-mediated condition or disease” and related phrases and terms refer to a condition characterized by inappropriate, e.g., less than or greater than normal, CXCR3 activity. Inappropriate CXCR3 activity might arise as the result of CXCR3 expression in cells which normally do not express CXCR3, increased CXCR3 expression (leading to, e.g., inflammatory and immunoregulatory disorders and diseases), or, decreased CXCR3 expression (leading to, e.g., certain cancers and angiogenic and vasculogenic-related disorders). Inappropriate CXCR3 functional activity might arise as the result of CXCR3 expression in cells which normally do not express CXCR3, increased CXCR3 expression (leading to, e.g., inflammatory and immunoregulatory disorders and diseases) or decreased CXCR3 expression. Inappropriate CXCR3 functional activity might also arise as the result of chemokine secretion by cells which normally do not secrete a CXC chemokine, increased chemokine expression (leading to, e.g., inflammatory and immunoregulatory disorders and diseases) or decreased chemokine expression. A CXCR3-mediated condition or disease may be completely or partially mediated by inappropriate CXCR3 functional activity. However, a CXCR3-mediated condition or disease is one in which modulation of CXCR3 results in some effect on the underlying condition or disease (e.g., a CXCR3 antagonist results in some improvement in patient well-being in at least some patients).

The term “therapeutically effective amount” means the amount of the subject compound that will elicit the biological or medical response of a tissue, system, animal or human that is being sought by the researcher, veterinarian, medical doctor or other clinician or that is sufficient to prevent development of or alleviate to some extent one or more of the symptoms of the disease being treated.

Diseases and conditions associated with inflammation, infection and cancer can be treated with the present compounds and compositions. In one group of embodiments, diseases or conditions, including chronic diseases, of humans or other species can be treated with inhibitors of CXCR3 function. These diseases or conditions include: (1) inflammatory or allergic diseases such as systemic anaphylaxis or hypersensitivity responses, drug allergies, insect sting allergies and food allergies; inflammatory bowel diseases, such as Crohn's disease, ulcerative colitis, ileitis and enteritis; vaginitis; psoriasis and inflammatory dermatoses such as dermatitis, eczema, atopic dermatitis, allergic contact dermatitis, urticaria; vasculitis; spondyloarthropathies; scleroderma; asthma and respiratory allergic diseases such as allergic rhinitis, hypersensitivity lung diseases, and the like, (2) autoimmune diseases, such as arthritis (rheumatoid and psoriatic), multiple sclerosis, systemic lupus erythematosus, type I diabetes, glomerulonephritis, and the like, (3) graft rejection (including allograft rejection and graft-v-host disease) and conditions associated therewith, and (4) other diseases in which undesired inflammatory responses are to be inhibited, e.g., atherosclerosis, myositis, neurodegenerative diseases (e.g., Alzheimer's disease), encephalitis, meningitis, hepatitis, nephritis, sepsis, sarcoidosis, conjunctivitis, otitis, chronic obstructive pulmonary disease, sinusitis and Behcet's syndrome. In another group of embodiments, diseases or conditions are treated with agonists of CXCR3 function. Examples of diseases to be treated with CXCR3 agonists include cancers, diseases in which angiogenesis or neovascularization play a role (neoplastic diseases, retinopathy and macular degeneration), infectious diseases and immunosuppressive diseases.

Preferably, the present methods are directed to the treatment or prevention of diseases or conditions selected from neurodegenerative diseases (e.g., Alzheimer's disease), multiple sclerosis, systemic lupus erythematosus, rheumatoid arthritis, atherosclerosis, encephalitis, meningitis, hepatitis, nephritis, sepsis, sarcoidosis, psoriasis, eczema, uticaria, type I diabetes, asthma, conjunctivitis, otitis, allergic rhinitis, chronic obstructive pulmonary disease, sinusitis, dermatitis, inflammatory bowel disease, ulcerative colitis, Crohn's disease, Behcet's syndrome, gout, cancer, viral infections (e.g., HIV), bacterial infections, and organ transplant conditions or skin transplant conditions. The term “organ transplant conditions” is meant to include bone marrow transplant conditions and solid organ (e.g., kidney, liver, lung, heart, pancreas or combination thereof) transplant conditions.

Diseases or conditions that can be treated with the present compounds and compositions include diseases commonly associated with (1) inflammatory or allergic diseases, (2) autoimmune diseases, (3) graft rejection and (4) other diseases in which undesired inflammatory responses are to be inhibited, as described above. For example, restenosis following a procedure such as balloon angioplasty, is commonly associated with atherosclerosis and can be treated with the present compounds and compositions.

Depending on the disease to be treated and the subject's condition, the compounds of the present invention may be administered by oral, parenteral (e.g., intramuscular, intraperitoneal, intravenous, ICV, intracisternal injection or infusion, subcutaneous injection, or implant), inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

In the treatment or prevention of conditions which require chemokine receptor modulation an appropriate dosage level will generally be about 0.001 to 100 mg per kg patient body weight per day which can be administered in single or multiple doses. Preferably, the dosage level will be about 0.01 to about 25 mg/kg per day; more preferably about 0.05 to about 10 mg/kg per day. A suitable dosage level may be about 0.01 to 25 mg/kg per day, about 0.05 to 10 mg/kg per day, or about 0.1 to 5 mg/kg per day. Within this range the dosage may be 0.005 to 0.05, 0.05 to 0.5 or 0.5 to 5.0 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The compounds of the present invention can be combined with other compounds having related utilities to treat or prevent inflammatory and immune disorders and diseases, including asthma and allergic diseases, as well as autoimmune pathologies such as rheumatoid arthritis and atherosclerosis, and those pathologies noted above. In many instances, compositions which include a compound of the invention and an alternative or second therapeutic agent have additive or synergistic effects when administered.

For example, in the treatment or prevention of inflammation, the present compounds may be used in conjunction or combination with an anti-inflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal anti-inflammatory agent, or a cytokine-suppressing anti-inflammatory agent, for example with a compound such as acetaminophen, aspirin, codeine, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextromethorphan; a diuretic; and a sedating or non-sedating antihistamine. Likewise, compounds of the present invention may be used in combination with other drugs that are used in the treatment/prevention/suppression or amelioration of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention. Examples of other active ingredients that may be combined with a compound of the present invention, either administered separately or in the same pharmaceutical compositions, include, but are not limited to: (a) VLA-4 antagonists, (b) steroids such as beclomethasone, methylprednisolone, betamethasone, prednisone, dexamethasone, and hydrocortisone; (c) immunosuppressants such as cyclosporine (cyclosporine A, Sandimmune®, Neoral®), tacrolimus (FK-506, Prograf®), rapamycin (sirolimus, Rapamune®) and other FK-506 type immunosuppressants, and mycophenolate, e.g., mycophenolate mofetil (CellCept®); (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (e) non-steroidal anti-asthmatics such as β2-agonists (terbutaline, metaproterenol, fenoterol, isoetharine, albuterol, bitolterol, and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal anti-inflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors such as celecoxib (Celebrex®) and rofecoxib (Vioxx®); (h) inhibitors of phosphodiesterase type IV (PDE-IV); (i) gold compounds such as auranofin and aurothioglucose, (j) inhibitors of phosphodiesterase type IV (PDE-IV); (k) other antagonists of the chemokine receptors, especially CCR1, CCR2, CCR3, CCR5, CCR6, CCR8 and CCR10; (1) cholesterol lowering agents such as HMG-CoA reductase inhibitors (lovastatin, simvastatin and pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (m) anti-diabetic agents such as insulin, sulfonylureas, biguamides (metformin), α-glucosidase inhibitors (acarbose) and glitazones (troglitazone and pioglitazone); (n) preparations of interferon beta (interferon β-1α, interferon β-1β); (O) etanercept (Enbrel®), (p) antibody therapies such as orthoclone (OKT3), daclizumab (Zenapax®), infliximab (Remicade®), basiliximab (Simulect®) and anti-CD40 ligand antibodies (e.g., MRP-1); and (q) other compounds such as 5-aminosalicylic acid and prodrugs thereof, hydroxychloroquine, D-penicillamine, antimetabolites such as azathioprene and 6-mercaptopurine, and cytotoxic cancer chemotherapeutic agents. The weight ratio of the compound of the present invention to the second active ingredient may be varied and will depend upon the effective dose of each ingredient. Generally, an effective dose of each will be used. Thus, for example, when a compound of the present invention is combined with an NSAID the weight ratio of the compound of the present invention to the NSAID will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

Immunosuppressants within the scope of the present invention further include, but are not limited to, leflunomide, RAD001, ERL080, FTY720, CTLA-4, antibody therapies such as orthoclone (OKT3), daclizumab (Zenapax®) and basiliximab (Simulect®), and antithymocyte globulins such as thymoglobulins.

In particularly preferred embodiments, the present methods are directed to the treatment or prevention of multiple sclerosis using a compound of the invention either alone or in combination with a second therapeutic agent selected from betaseron, avonex, azathioprene (Imurek®, Imuran®), capoxone, prednisolone and cyclophosphamide. When used in combination, the practitioner can administer a combination of the therapeutic agents, or administration can be sequential.

In still other particularly preferred embodiments, the present methods are directed to the treatment or prevention of rheumatoid arthritis, wherein the compound of the invention is administered either alone or in combination with a second therapeutic agent selected from the group consisting of methotrexate, sulfasalazine, hydroxychloroquine, cyclosporine A, D-penicillamine, infliximab (Remicade®), etanercept (Enbrel®), auranofin and aurothioglucose.

In yet other particularly preferred embodiments, the present methods are directed to the treatment or prevention of an organ transplant condition wherein the compound of the invention is used alone or in combination with a second therapeutic agent selected from the group consisting of cyclosporine A, FK-506, rapamycin, mycophenolate, prednisolone, azathioprene, cyclophosphamide and an antilymphocyte globulin.

### 5.6 Dosing of Compound 101 to Treat CXCR3-Mediated Conditions

In addition, aspects of the present invention are based on the discovery that compound 101, described in U.S. patent application Publication No. US 2002/0169159 A1, can be administered to a subject in doses that provide the subject with a therapeutically effective amount of a compound of the invention. Thus, the present invention provides methods of treating CXCR3-mediated conditions or diseases comprising administering to a subject having such a disease or condition a dose of a parent compound sufficient to provide the subject a therapeutically effective amount of one or more compounds of the invention. While not intending to be bound by any particular theory of operation, it is believed that appropriate dosing of a parent compound can provide a subject with an effective amount of a compound of the invention. For example, appropriate dosing of compound 101 according to the methods herein can be used to provide a subject with an effective amount of compound 103. In another embodiment, certain doses disclosed herein can be used to treat or prevent disease while avoiding or reducing unwanted or adverse side effects.

In certain methods of the invention, a dose of a parent compound such as compound 101 is given to the subject to achieve a therapeutically effective plasma concentration of a compound of the invention. In preferred embodiments, the therapeutically effective plasma concentration of a compound of the invention is from about 1 nM to about 10 μM, from about 10 nM to about 1 μM or from about 100 nM to about 1000 μM.

Further aspects of the invention are based on the observation that the therapeutic effectiveness of compound 101 is in part due to the characteristic pharmacokinetic profile associated with administration of compound 101 and resulting metabolites of compound 101. Thus, in methods of the invention a therapeutically effective amount of a parent compound, is administered to a patient in a manner such that a characteristic pharmacokinetic profile for either the parent compound or the active compound is obtained, thereby treating a CXCR3-mediated condition or disease. The preferred parent compound is compound 101.

Following administration of compound 101, plasma concentrations of both compound 101 and metabolites thereof are detected in mice, rats, dogs and humans. In some species, the levels of certain metabolites may exceed the plasma concentrations of compound 101, while in others the levels of certain metabolites may be comparable to or lower than the plasma concentrations of compound 101.

### 6. EXAMPLES

Reagents and solvents used below can be obtained from commercial sources such as Aldrich Chemical Co. (Milwaukee, Wis., USA). ¹H-NMR spectra were recorded on a Varian Gemini 400 MHZ NMR spectrometer. Significant peaks are tabulated in the order: number of protons, multiplicity (s, singlet; d, doublet; t, triplet; q, quartet; m, multiplet; br s, broad singlet) and coupling constant(s) in Hertz (Hz). Electrospray ionization (ESI) mass spectrometry analysis was conducted on a Hewlett-Packard 1100 MSD electrospray mass spectrometer using the HP1 100 HPLC for sample delivery. Mass spectrometry results are reported as the ratio of mass over charge. Each compound was dissolved in methanol at 0.1 mg/mL and 1 microliter was infused with the delivery solvent into the mass spectrometer, which scanned from 100 to 1500 daltons. Each compound could be analyzed in the positive ESI mode, using 1:1 acetonitrile/water with 1% acetic acid as the delivery solvent. Each compound could also be analyzed in the negative ESI mode, using 2 mM NH₄OAc in acetonitrile/water as delivery solvent.

### Example 1

### Synthesis of Compound 101

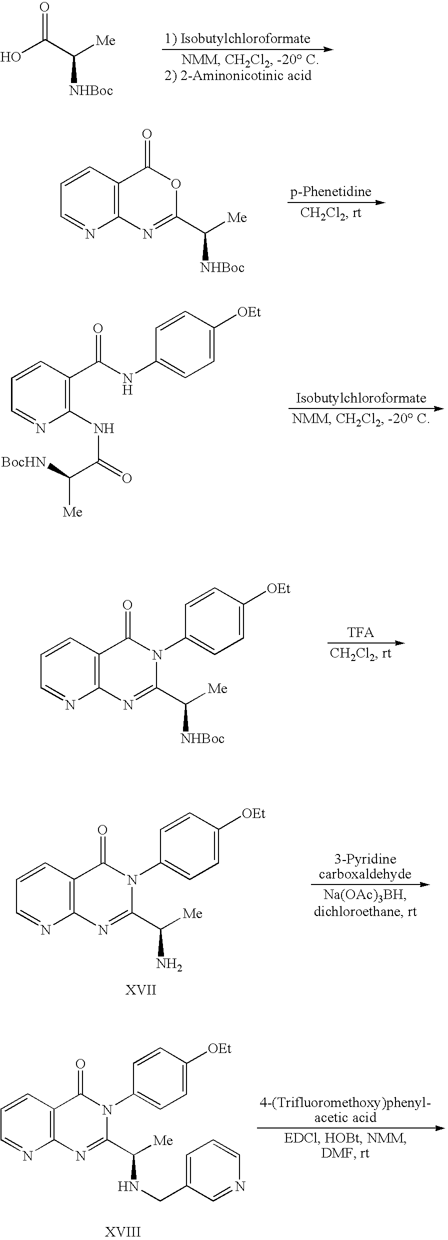

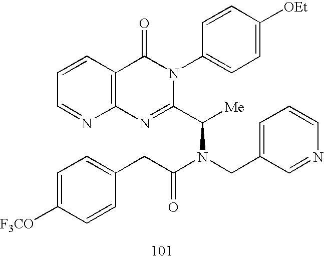

(1-N-BOC-aminoethyl)-3-(4-ethoxyphenyl)-2-{(1R)-1-[(pyridin-3-yl-methyl)-amino]-ethyl}-3H-pyrido[2,3-d]pyrimidin-4-one (XVII precursor).

To a 3 L round bottom flask equipped with addition funnel, mechanical stirrer and temperature probe was added 102.60 g (542.26 mmol) of N-(tert-butoxycarbonyl)-D-alanine in 1.2 L of dichloromethane (DCM) under a nitrogen atmosphere. The solution was cooled to −20° C. and 150.00 ml (1364.31 mmol) of N-methyl morpholine added followed by the addition of a solution containing 140.1 ml (1084 mmol) of iso-butylchloroformate in 360 ml of DCM over 40 min. while maintaining the reaction temperature below −20° C. After complete addition, the reaction was allowed to stir for 45 min. and 75.00 g (542.97 mmol) of 2-aminonicotinic acid added. The reaction was allowed to warm to room temperature overnight. The reaction was diluted with 1.5 L DCM and washed with 1.0 N hydrochloric acid (2×750 ml) and brine (1×500 ml). The organic phase was dried over magnesium sulfate, filtered, and concentrated in vacuo to give 175.0 g of a yellow-orange oil. The material was used without further purification in the next step.

A solution containing the crude material from above dissolved in 2 L DCM was cooled to −20° C. under a nitrogen atmosphere and 69.00 ml (535.68 mmol) of p-phenetidine was added over 5 minutes. After stirring with gradual warming to 0° C. the reaction mixture was transferred to a separatory funnel and washed with 1.0 N hydrochloric acid (2×500 ml), saturated sodium bicarbonate solution (2×1 L), and brine (1×1 L). The organic phase was dried over magnesium sulfate, filtered, and concentrated in vacuo to give 175.2 g of crude bis-amide. The material was used without further purification in the next step.

A solution containing the crude bis-amide prepared above in 2.3 L of DCM and 50.0 ml (454.7 mmol) of N-methyl morpholine was cooled to −20° C. under a nitrogen atmosphere and 53.0 ml (408.6 mmol) of iso-butylchloroformate was added dropwise over a period of 5 minutes. Upon completed addition of the chloroformate, HPLC analysis indicated no bis-amide remained. The reaction mixture was transferred to a separatory funnel and washed with 1.0 N hydrochloric acid (2×1 L), saturated bicarbonate solution (1×1 L), and brine (1×1 L). The organic phase was dried over magnesium sulfate, filtered, and concentrated in vacuo to give 205 g of a brown, viscous oil. This product was dissolved in 500 ml of methyl tert-butyl ether and allowed to stir until the product began to precipitate from the solution. Heptane was then added (1000 ml) and stirring continued. The resulting precipitate was collected by filtration, washed with heptane, and dried to afford 78.9 g of product as an off-white solid. ¹H NMR (CDCl₃) δ 8.99 (dd, J₁=2.0 Hz, J₂=4.4 Hz, 1H), 8.60 (dd, J₁=2.0 Hz, J₂=8.0 Hz, 1H), 7.44 (dd, J₁=4.4 Hz, J₂=8.0 Hz, 1H), 7.33 (dd, J₁=1.6 Hz, J₂=8.8 Hz, 1H), 7.16 (dd, J₁=2.8 Hz, J₂=8.8 Hz, 1H), 7.20 (dd, J₁=2.4 Hz, J₂=8.8 Hz, 1H), 7.04 (dd, J₁=2.8 Hz, J₂=8.8 Hz, 1H), 5.80 (d, J=8.8 Hz, 1H), 4.63-4.70 (m, 1H), 4.06-4.13 (q, J=7.2 Hz, 2H), 1.46 (t, J=7.2 Hz, 3H), 1.40 (s, 9H), 1.31 (d, J=6.8 Hz, 3H), ppm.

Intermediate XVII

To a solution containing 77.00 g (187.59 mmol) of the compound prepared above in 2.1 L of DCM was added 290 mL trifluoroacetic acid. The reaction was allowed to stir for 3.5 h at room temperature then concentrated in vacuo. The concentrate was dissolved in 1.4 L DCM and washed with 1.0 N hydrochloric acid (3×500 ml). The combined aqueous washes were made alkaline by addition of concentrated ammonium hydroxide until pH=10. The resulting cloudy solution was extracted with DCM (2×700 ml) and the combined organic extracts dried over magnesium sulfate, filtered, and concentrated in vacuo to afford 58.40 g of product as a tan solid. ¹H NMR (DMSO-d₆) δ 8.94 (dd, J₁=2.0 Hz, J₂=4.8 Hz, 1H), 8.44 (dd, J₁=2.0 Hz, J₂=8.0 Hz, 1H), 7.49 (dd, J₁=4.8 Hz, J₂=8.0 Hz, 1H), 7.34-7.39 (m, 2H), 7.04-7.10 (m, 2H), 4.08 (q, J=6.8 Hz, 2H), 3.52 (q, J=6.4 Hz, 1H), 1.94 (br s, 2H), 1.34 (t, J=6.8 Hz, 3H), 1.15 (d, J=6.4 Hz, 3H) ppm.

Intermediate XVIII

To a solution containing 57.70 g (185.92 mmol) of intermediate XVII prepared above dissolved in 1.7 L of dichloroethane was added 18.5 ml (196.04 mmol) pyridine carboxaldehyde followed by 55.20 g (260.45 mmol) of sodium triacetoxy borohydride. The reaction was allowed to stir at room temperature overnight. The reaction was diluted with 1 L of DCM and washed with 1.0 M ammonium hydroxide (2×500 ml). The organic phase was dried over magnesium sulfate, filtered, and concentrated in vacuo to afford 79.20 g of product as a pale yellow solid. ¹H NMR (DMSO-d₆) δ8.96-8.98 (m, 1H), 8.42-8.48 (m, 1H), 8.45 (br s, 1H), 8.37 (d, J=4.8 Hz, 1H), 7.64 (d, J=8.0 Hz, 1H), 7.52 (dd, J₁=4.8 Hz, J₂=8.0 Hz, 1H), 7.33 (dd, J₁=2.4 Hz, J₂=8.4 Hz, 1H), 7.24 (dd, J₁=4.8 Hz, J₂=8.0 Hz, 1H), 7.14 (dd, J₁=2.4 Hz, J₂=8.4 Hz, 1H), 6.99 (dd, J₁=2.8 Hz, J₂=8.4 Hz, 1H), 6.83 (dd, J₁=2.8 Hz, J₂=8.8 Hz, 1H), 3.97-4.10 (m, 1H), 3.87 (s, 1H), 3.72 (d, J=14.0 Hz, 1H), 3.52 (d, J=13.6 Hz, 1H), 3.28 (q, J=6.4 Hz, 1H), 1.31 (t, J=7.2 Hz, 3H), 1.17 (d, J=6.4 Hz, 3H) ppm.

Compound 101

To a solution containing 54.00 g (245.29 mmol) of 4-(trifluoromethoxy) phenylacetic acid in 1.1 L of DMF was added 61.30 g (319.77 mmol) of EDCI, 43.20 g (319.69 mmol) HOBT and 42.00 ml (382.01 mmol) of N-methyl morpholine. After stirring for 30 min., 74.60 g (185.82 mmol) of intermediate XVIII was added. The reaction was allowed to stir at room temperature overnight. The reaction was diluted with 3 L DCM and washed with water (2×3 L), saturated sodium bicarbonate solution (2×2 L), and brine (1×2 L). The organic extract was dried over magnesium sulfate, filtered, and concentrated in vacuo to afford 121.7 g of a yellow solid. The solids were triturated with 700 ml of methyl tert-butyl ether, collected by filtration, rinsed, and dried to afford 88.46 g of product as an off-white solid.

The product was recrystallized from 10% ethyl acetate in heptane to afford a colorless, microcrystalline (small needles) solid, m.p. 161.2° C. ¹H NMR (T=120° C.; DMSO-d₆) δ9.01 (dd, J₁=1.6 Hz, J₂=4.4 Hz, 1H), 8.46 (dd, J₁=2.0 Hz, J₂=7.6 Hz, 1H), 8.35 (m, 2H), 7.57 (dd, J₁=4.8 Hz, J₂=8.4 Hz, 1H), 7.55 (d, J=8.0 Hz, 1H), 7.43 (d, J=8.0 Hz, 1H), 7.06-7.22 (m, 7H), 5.28 (q, J=6.0 Hz, 1H), 4.76 (br s, 2H), 4.13 (q, J=6.8 Hz, 2H), 3.48 (br s, 0.5-1H [H₂O]), 2.91 (br s, 2H), 1.42 (d, J=6.8 Hz, 3H), 1.36 (t, J=6.8 Hz), ppm.

HPLC>99%, chiral HPLC>96% ee). MS (ESI, positive mode): 626 (MH⁺).

### Example 2

This example illustrates the discovery of metabolites of compound 101 that form the basis of the compounds of the invention.

Compound 101 was contacted with microsomal fractions from Sprague Dawley rat, dog (beagle), rhesus monkey and human liver to detect and identify resulting metabolites. In particular, Compound 101 in PBS (10 μM) was incubated in a mixture of microsomal protein (1 mg/mL), EDTA (1.0 mM) and NADPH (5 mM) at 37° C. for a total of 105 min. The reaction was stopped by the addition of acetonitrile containing 0.2% formic acid (400 μL). Samples were centrifuged and the supernatant was subjected to LCMS analysis.

Compound 103, the pyridyl-N-oxide product of compound 101, was detected following contact with the microsomal fractions from all species. Compound 105, the deethylation product of compound 101, was also detected following contact with the microsomal fractions from all species. With rat liver microsomal fractions, approximately equal amounts of compounds 103 and 105 were detected. With dog, monkey and human microsomal fractions, more compound 103 than compound 105 was detected. With human microsomal fractions, an amount of compound 107, the deethylation and pyridyl-N-oxide product of compound 101, was also detected.

Compound 101 was also incubated with intact dog and monkey liver cells (cryopreserved hepatocytes). Following incubation, the formation of compound 109, the glucuronide adduct of compound 105, was detected.

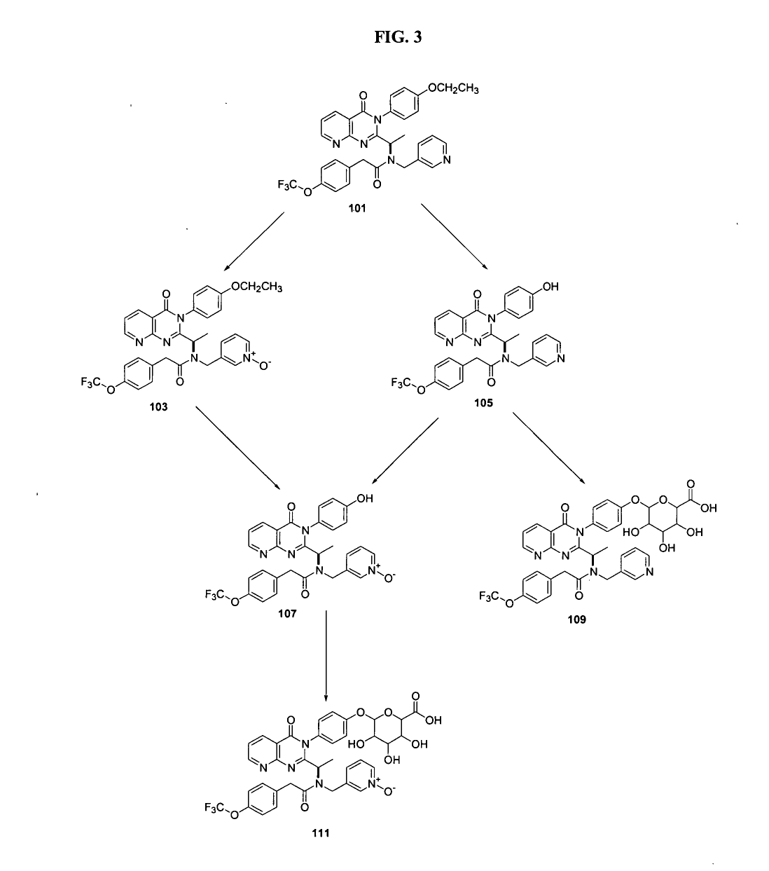

In addition, compound 101 was administered intravenously or orally to bile duct cannulated male Sprague Dawley rats. Urine and bile samples were collected over a period of 24 hours and analyzed by liquid chromatography/mass spectrometry. Compound 101 was detected in a small amount (<1% of the administered dose) in urine and in bile. In urine, compound 103, the pyridyl-N-oxide of compound 101, was detected. Five metabolites, compounds 103, 105, 107, 109 and 111, were all detected in bile excretions; compound 111 was detected in a trace amount in bile. A proposed metabolic pathway for compound 101 is provided at FIG. 3.

Overall, N-oxidation and O-deethylation occurred to similar extents. The O-deethyl metabolite was detected mainly as a glucuronide conjugate, compound 109. The N-oxidized O-deethyl metabolite, compound 107, was detected in bile in significant amounts when compared to compounds 103 and 105. Only trace amounts of the glucuronide of the N-oxidized O-deethyl metabolite, compound 109, were detected. The detected metabolites, and the relative amounts thereof, in urine and bile agree with the metabolites detected with rat liver microsomal fractions and rat hepatocytes in vitro as discussed above.

The observed metabolic forms of compound 101 are summarized in the table below.

Metabolic Forms of Compound 101

| Compound | Human | Monkey | Dog | Rat (in vitro) | Rat (in vivo) |
|---|---|---|---|---|---|
| 101 | | | | | <1% |
| 103 | major | major | major | major | urine & bile |
| 105 | minor | minor | minor | major | bile |
| 107 | minor | minor | | | bile |
| 109 | | minor | minor | | bile |
| 111 | | | | | bile (traces) |

### Example 3

### Human Metabolism of Compound 101

This example demonstrates the metabolism of compound 101 by human P450 3A4.

Compound 101 (1 μM) was incubated with human liver microsomes in the presence and absence of specific inhibitors of human cytochrome P450 isoforms 1A2, 2A6, 2C9, 2E1 and 3A4. Ketoconazole (1 μM), a specific inhibitor of cytochrome P450 3A4 abolished the formation of the pyridyl-N-oxide metabolite, compound 103, the predominant human metabolite of compound 101. The inhibition was greater than 90%. Minimal to no inhibition of metabolism of compound 101 was observed with the other cytochrome p450 inhibitors.

Further experiments verified that compound 101 produced little or no inhibition or induction of any cytochrome p450 isoform, including 3A4, suggesting little or no drug-drug interactions from administering compound 101 to human subjects due to cytochrome p450 interactions.

### Example 4

This example illustrates a CXCR3 binding assay that can be used for evaluating compounds of the present invention.

Unless otherwise noted, all reagents used are available from commercial sources (e.g., Sigma). Test compounds can be diluted in DMSO to a concentration that is 40-times the intended final assay concentration; 5 μL can be transferred to each well of a 96-well flat-bottomed polypropylene plate (e.g., from Greiner, Inc.). CXCR3-expressing cells can be resuspended in assay buffer (25 mM Hepes, 80 mM NaCl, 1 mM CaCl₂, 5 mM MgCl₂, 0.2% bovine serum albumin, pH 7.1, stored at 4° C.) at 5 million cells per mL; 100 μL of this cell suspension can then be transferred to each well of a 96-well plate containing the diluted test compounds. ¹²⁵I-labeled chemokine (purchased from commercial sources, e.g., Amersham, PE Life Sciences) can be diluted in assay buffer to a concentration of approximately 60 pM; 100 μL of this chemokine solution can be transferred to each well of a 96-well plate containing compounds and cell suspension. The plates can be sealed with commercially available foil plate seals (e.g., from E&K Scientific), and stored at 4° C. for 2 to 4 h, shaking gently. At the end of this incubation period, the contents of the assay plates can be transferred to GF/B filter plates (Packard) that have been pre-coated by dipping into a solution containing 0.3% polyethyleneimine (Sigma), using a cell harvester (Packard), and washing twice with wash buffer (25 mM Hepes, 500 mM NaCl, 1 mM CaCl₂, 5 mM MgCl₂, pH 7.1, stored at room temperature). The filter plates can be sealed on the bottom with plate seals (Packard), 50 μL of Microscint-20 scintillation fluid (Packard) can be added to each well, and the top of the plates can be sealed with clear plastic (TopSeal A, Packard). The plates can be counted on a scintillation counter, such as a Packard TopCount. To measure non-specific binding, 4 wells containing unlabeled “cold” chemokine can be included on each 96-well plate. To measure maximum binding, 4 wells containing 5 μL of DMSO, 100 μL of cell suspension and 100 μL of ¹²⁵I-labeled chemokine solution can be included on each 96-well plate. Data can be analyzed using commercially available software (e.g., Excel from Microsoft, Prism from GraphPad Software Inc.).

Other assays may be used to identify compounds that modulate CXCR3 chemokine receptor activity, for example, binding assays (see, e.g., Weng et al. (1998) J. Biol. Chem. 273:18288-18291, Campbell et al. (1998) J. Cell Biol. 141:1053-1059, Endres et al. (1999) J. Exp. Med. 189:1993-1998 and Ng et al. (1999) J. Med. Chem. 42:4680-4694), calcium flux assays (see, e.g., Wang et al. (2000) Mol. Pharm. 57:1190-1198 and Rabin et al. (1999) J. Immunol. 162:3840-3850) and chemotaxis assays (see, e.g., Albanesi et al. (2000) J. Immunol. 165:1395-1402 and Loetscher et al. (1998) Eur. J. Immunol. 28:3696-3705).

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A compound having the formula (I): 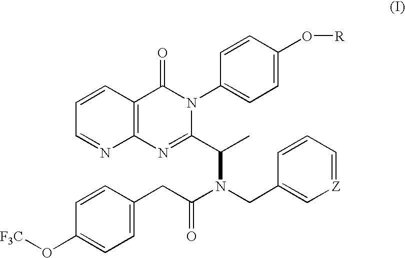
wherein Z is N or N⁺—O⁻; and R is hydrogen, (C₁-C₂₀)alkyl, (C₂-C₂₀)heteroalkyl, heteroaryl, aryl, heteroaryl(C₁-C₆)alkyl, heteroaryl(C₂-C₆)heteroalkyl, aryl(C₁-C₆)alkyl or aryl(C₂-C₆)heteroalkyl; with the proviso that when Z is N, R is not (C₁-C₂)alkyl, (C₂)heteroalkyl or aryl(C₁-C₃)alkyl.

2. The compound of claim 1 wherein Z is N.

3. The compound of claim 1 wherein Z is N⁺—O⁻.

4. The compound of claim 1 wherein R is hydrogen.

5. The compound of claim 1 wherein R is a saccharide or a saccharide derivative.

6. The compound of claim 1 wherein R is glucuronide.

7. The compound of claim 1 selected from the group consisting of:

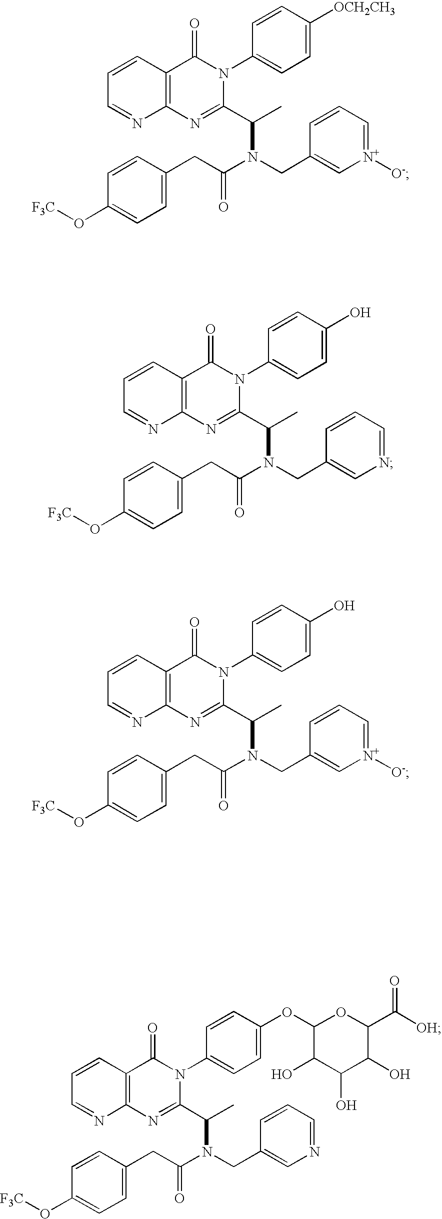

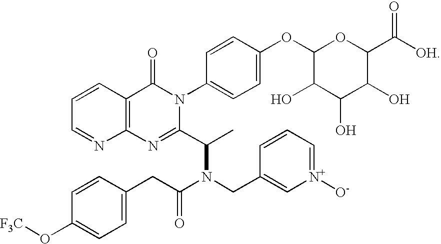

8. The compound of claim 7 that is

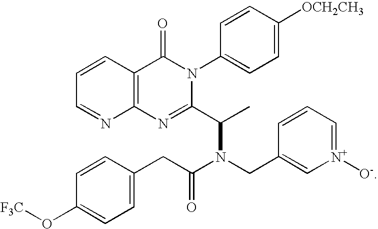

9. A pharmaceutical composition comprising the compound of claim 1 or 7 and a pharmaceutically acceptable carrier.

10. A method of treating an CXCR3-mediated condition in a subject, said method comprising administering to the subject a therapeutically effective amount of the compound of claim 1.

11. A method in accordance with claim 10, wherein said CXCR3-mediated condition is selected from the group consisting of neurodegenerative diseases, multiple sclerosis, systemic lupus erythematosus, rheumatoid arthritis, atherosclerosis, encephalitis, meningitis, hepatitis, nephritis, sepsis, sarcoidosis, psoriasis, eczema, uticaria, type I diabetes, asthma, conjunctivitis, otitis, allergic rhinitis, chronic obstructive pulmonary disease, sinusitis, dermatitis, inflammatory bowel disease, Behcet's syndrome, gout, cancer, viral infections, bacterial infections, organ transplant conditions and skin transplant conditions.

12. A method of treating an CXCR3-mediated condition in a subject, said method comprising administering to the subject an amount of a parent compound according to formula (II): 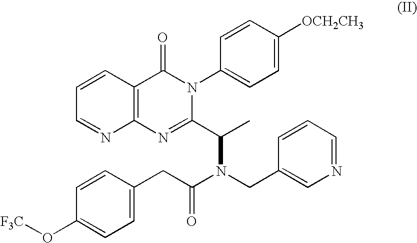
that provides the subject a therapeutically effective amount of the compound of claim 1.

13. The method of claim 12 wherein the parent compound according to formula (II) provides the subject a therapeutically effective amount of

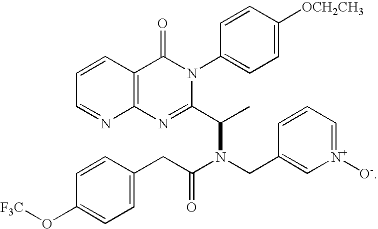

14. A method in accordance with claim 13, wherein said CXCR3-mediated condition is selected from the group consisting of neurodegenerative diseases, multiple sclerosis, systemic lupus erythematosus, rheumatoid arthritis, atherosclerosis, encephalitis, meningitis, hepatitis, nephritis, sepsis, sarcoidosis, psoriasis, eczema, uticaria, type I diabetes, asthma, conjunctivitis, otitis, allergic rhinitis, chronic obstructive pulmonary disease, sinusitis, dermatitis, inflammatory bowel disease, Behcet's syndrome, gout, cancer, viral infections, bacterial infections, organ transplant conditions and skin transplant conditions.

15. A method in accordance with claim 13, wherein said CXCR3-mediated condition is psoriasis, inflammatory bowel disease, multiple sclerosis, rheumatoid arthritis, asthma or an organ transplant condition.

16. The method of claim 15 wherein CXCR3-mediated condition is psoriasis.

17. The method of claim 15 wherein said CXCR3-mediated condition is inflammatory bowel disease.

18. The method of claim 17 wherein said inflammatory bowel disease is ulcerative colitis or Crohn's disease.

19. The method of claim 15 wherein CXCR3-mediated condition is multiple sclerosis, rheumatoid arthritis or an organ transplant condition.

20. A method in accordance with claim 13, wherein said subject is a human.

21. A method in accordance with claim 13 wherein said compound modulates CXCR3.

22. A method in accordance with claim 21 wherein said compound is a CXCR3 antagonist.

23. A method of treating or preventing a condition in a subject, said method comprising administering to the subject a therapeutically or prophylactically effective amount of the compound of claim 1, wherein said condition is selected from the group consisting of neurodegenerative diseases, multiple sclerosis, systemic lupus erythematosus, rheumatoid arthritis, atherosclerosis, encephalitis, meningitis, hepatitis, nephritis, sepsis, sarcoidosis, psoriasis, eczema, uticaria, type I diabetes, asthma, conjunctivitis, otitis, allergic rhinitis, chronic obstructive pulmonary disease, sinusitis, dermatitis, inflammatory bowel disease, Behcet's syndrome, gout, cancer, viral infections, bacterial infections, organ transplant conditions and skin transplant conditions.

24. The method of claim 23 that comprises administering to the subject an amount of a parent compound according to formula (II): 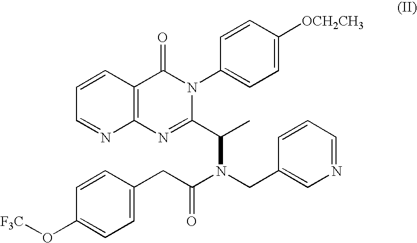
that provides the subject a therapeutically or prophylactically effective amount of the compound of claim 1.
